(19) United States
(12) Patent Application Publication
(10) Pub. No.: US 2013/0204190 A1
(43) Pub. Date: Aug. 8, 2013

(54) INTRAVENOUS SPLINT COVER AND ASSOCIATED METHODS

(71) Applicants: Marie-Andrea I. Wilborn, Melbourne, FL (US); Marie Therese R. Fongemie, Melbourne, FL (US)

(72) Inventors: Marie-Andrea I. Wilborn, Melbourne, FL (US); Marie Therese R. Fongemie, Melbourne, FL (US)

(21) Appl. No.: 13/367,498

(22) Filed: Feb. 7, 2012

(51) Int. Cl. A61M 5/32

(52) U.S. Cl. 604/180

(57) ABSTRACT

An intravenous splint cover may include a main body and a pair of opposing securing members connected to the main body. The intravenous splint cover may also include a splinting member carried by the main body to create an elevated portion of the main body. Bottom portions of the pair of opposing securing members and the main body that does not carry the splinting member may have an adhesive material applied thereto. The splinting member may extend along a medial portion of the bottom portion of the main body substantially between an entire width thereof. The intravenous splint cover according to the present invention advantageously secures an IV site of a patient while allowing the extremity of the patient with IV is located to remain mobile.

### FIELD OF THE INVENTION

The present invention relates to the medical field and, more specifically, to the field of intravenous splint covers and associated methods.

### BACKGROUND OF THE INVENTION

It can sometimes be uncomfortable when patients require medical treatment that involves an intravenous line (IV). Although IV medical treatment is very common, and is a routine way to deliver medication, the starting of an IV is generally a process that many patients do not like, i.e., being stuck with a needle. In some instances, an IV may come out, i.e., require reinsertion, thereby causing discomfort to the patient, and exposing the patient to possible infection. Another issue that may arise is occlusion of the IV due to the bending of an IV tube.

There have been attempts to prevent an IV from coming out such as, for example, immobilizing the patient's extremity where the IV is positioned. U.S. Pat. No. 4,425,913 to Lewis discloses a splint for supporting the hand, wrist and forearm of a patient when the patient is connected to an IV. The splint incorporates a substantially rigid molded body. Accordingly, use of the splint disclosed in the Lewis '913 patent immobilizing a patient's extremity and can be uncomfortable for the patient. U.S. Pat. No. 3,776,225 to Lonardo also discloses a splint for supporting and maintaining a patient's forearm immobile while receiving an IV injection. Similar to the Lewis '913 patent, the Lonardo '225 patent is directed to immobilizing the patient's extremity and suffers from the same deficiency. Further, in cases where a patient may have edema, such devices do not stretch, which may cause fluid collection and clotting, which can, in turn, be problematic.

U.S. Pat. No. 7,182,088 to Jenkins discloses yet another arm immobilizer to be used when a patient is receiving IV treatments. The immobilizer described in the Jenkins '088 patent includes a closable sleeve of a compressible material which can be placed around the patient's arm above and below the elbow joint with an opening on the inside of the arm position to give access to the IV site. Although the device described in the Jenkins '088 patent provides for smaller movements of the patient's arm, it is still designed to prevent excesses flexure movement of the elbow. Further, this device uses metal and plastic materials in its construction. Accordingly, such device can be uncomfortable for the patient to wear on receiving IV treatments.

U.S. Pat. No. 7,799,001 to Bierman discloses a catheter securement device that holds a medical article upon the body of the patient and inhibits longitudinal movement of the medical article. The device disclosed in the Bierman '001 patent includes a central channel into which a portion of the IV may be inserted. Such a device may, however, be uncomfortable for patient, and may still inhibit flexural movement of the patient's extremity. Further, the device illustrated in the Bierman '001 patent may be expensive to manufacture.

U.S. Pat. No. 3,196,870 to Sprecher et al. discloses a limb immobilizer for intravenous feeding. This immobilizer may be used to cause the limb of the patient to remain straight and immobile while receiving IV treatments. More particularly, the illustrated version of Sprecher et al. '870 patent is directed to immobilizing the upper and lower arms of a human to prevent movement of the elbow joint, thereby facilitating intravenous administration of food, blood or other fluids. This device is made of uncomfortable materials, such as metals and plastics. Such a system depicts the very problem that needs to be addressed, i.e., immobilization of the extremities of the patient, thereby causing discomfort.

The devices described above, and other devices that may be known in the art generally suffer from the same deficiency, i.e., the devices prevent the patient from being able to move their extremity. Such restriction on movement also interferes with activities of daily living on the patient. Patients that are uncomfortable while receiving IV treatments may not respond as well to treatment as patients who are comfortable. Accordingly, a need exists for an intravenous splint cover that does not restrict movement of the patient and that allows the patient to engage in activities of daily living. There also exists a need for an intravenous splint cover that simultaneously secures an IV site so as to prevent any need for reinsertion of an IV.

### SUMMARY OF THE INVENTION

The intravenous splint cover according to an embodiment of the present invention advantageously contours to the patient's extremity where the IV is located so that the patient may maintain mobility and flexibility of the extremity while receiving IV treatments. The intravenous splint cover according to an embodiment of the present invention also advantageously does not immobilize extremities or joints of the patient and promotes comfort of the patient while receiving IV treatments. The intravenous splint cover according to an embodiment of the present invention further advantageously conceals the IV site, thereby directing the patient's attention away from the IV site, and preventing the patient from removing the IV. The present invention still further advantageously allows the IV site to remain accessible, and decreases the risk of occlusions of the IV catheter due to crimping and bending. Decreasing the risk of occlusions on the IV catheter also advantageously decreases the instances of reinsertion of the IV, thereby decreasing the risk of infection.

These and other features and advantages according to an embodiment of the present invention are provided by an intravenous (IV) splint cover that includes a main body having a bottom portion and a top portion. The IV splint cover may also include a pair of opposing securing members connected to and extending outwardly from the main body. Each of the pair of opposing securing members may have a bottom portion and a top portion. The IV splint cover may further include a splinting member carried by the bottom portion of the main body and extending outwardly from the main body to create an elevated portion of the main body.

The bottom portions of the pair of opposing securing members may have an adhesive material applied thereto, and the bottom portion of the main body that does not carry the splinting member may also have an adhesive material applied thereto. The splinting member may extend along the bottom portion of the main body substantially between an entire width thereof, and may be carried by a medial portion of the main body. Therefore, the IV splint cover according to an embodiment of the present invention may advantageously secure an IV on the extremity of the patient while simultaneously providing mobility to the extremity of the patient where the IV is positioned and allowing for ready viewing of the IV by a medical professional, as necessary.

Each of the pair of opposing securing members may include a respective pair of spaced apart securing members. Each of the respective pair of spaced apart securing members may extend substantially parallel to one another. The splinting member may be raised along a medial portion of the main body and may taper downwardly towards side portions of the main body. The IV splint cover according to an embodiment of the present invention may further include an adhesive cover member to be removeably positioned over the main body, the securing members and the splinting member. The adhesive cover member may be provided by a pair of adhesive cover members. A first one of the pair of adhesive cover members may extend from an end portion of one of the securing members to a medial portion of the splinting member, and a second one of the pair of adhesive cover members extends from an end portion of a second one of the securing member to the medial portion of the splinting member.

The adhesive material that may be applied to the bottom portions of the securing members may have stronger adhesive properties than the adhesive material that may be applied to the main body. This advantageously provides the ability for the IV splint cover to be readily secured to the patient so as to provide enhanced adhesive properties to the securing members to, in turn, provide for enhanced ability of the IV splint cover to remain applied over the IV site. The main body and the pair of opposing securing members may be integrally formed as a monolithic unit. This advantageously enhances manufacturing efficiency of the IV splint cover and decreases the cost of manufacturing. The main body and the pair of opposing securing members may be provided by a substantially flexible material, a biodegradable material, a hypoallergenic material, and/or a latex-free material.

A method aspect of the present invention is for using an IV splint cover. The method may include exposing the bottom portions of the main body, the pair of opposing securing members and the splinting member. The method may further include applying the splinting member to an IV site on a patient. The method may still further include securing the splinting member to the IV site by affixing the securing members to an extremity of the patient. This is preferably performed so that the main body of the IV splint cover and the opposing securing member are applied to the extremity of the patient where the IV is positioned so as not to immobilize the extremity and so that the IV site is readily accessible.

### BRIEF DESCRIPTION OF THE DRAWINGS

FIG. 1 is a bottom perspective view of an intravenous splint cover according to the present invention.

FIG. 2 is a top perspective view of the intravenous splint cover illustrated in FIG. 1.

FIG. 3 is a bottom plan view of the intravenous splint cover illustrated in FIG. 1.

FIG. 4 is a top plan view of the intravenous splint cover illustrated in FIG. 1.

FIG. 5 is a right-side elevation view of the intravenous splint cover illustrated in FIG. 1.

FIG. 6 is a left-side elevation view of the intravenous splint cover illustrated in FIG. 1.

FIG. 7 is a front elevation view of the intravenous splint cover illustrated in FIG. 1.

FIG. 8 is a rear elevation view of the intravenous splint cover illustrated in FIG. 1.

FIG. 9 is an environmental view of the intravenous splint cover illustrated in FIG. 1 as it is being positioned over an intravenous site on an extremity of a patient.

FIG. 10 is a perspective view of an intravenous splint cover according to an embodiment of the present invention showing a pair of adhesive cover-members positioned over a bottom surface portion thereof.

FIG. 11 is a perspective view of an intravenous splint cover according to an embodiment of the present invention.

FIG. 12 is a perspective view of the intravenous splint cover illustrated in FIG. 11 showing securing members being connected to one another.

### DETAILED DESCRIPTION OF THE PREFERRED EMBODIMENT

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout, and prime and multiple prime notations, if used, refer to similar elements in alternate embodiments.

In this detailed description of the present invention, a person skilled in the art should note that directional terms, such as “above,” “below,” “upper,” “lower,” and other like terms are used for the convenience of the reader in reference to the drawings and the accompanying descriptions. Also, a person skilled in the art should notice this description may contain other terminology to convey position, orientation, and direction without departing from the principles of the present invention.

Further, throughout this specification, the invention is referred to as an intravenous splint cover 20. Intravenous may sometimes be abbreviated as IV, and is not meant to be limiting in any way to the invention, as defined by the claims appended hereto. Throughout this disclosure, the intravenous splint cover 20 may be referred to as the IV splint cover, the splint cover, the system, the device, the apparatus or the invention. Alternate references to the intravenous splint cover 20 in this disclosure are not meant to be limiting in any way. Those skilled in the art will appreciate that an IV line may be positioned anywhere on a patient and, although the appended drawings illustrate an IV site being positioned on an arm of a patient, the present invention advantageously may be used regardless of the positioning of the IV line on the patient. The present invention is also not meant to be limited to use in connection with IV ones. Those skilled in the medical field will appreciate that the present invention can advantageously be used in connection with arterial lines and for dialysis lines, as well as other known treatments.

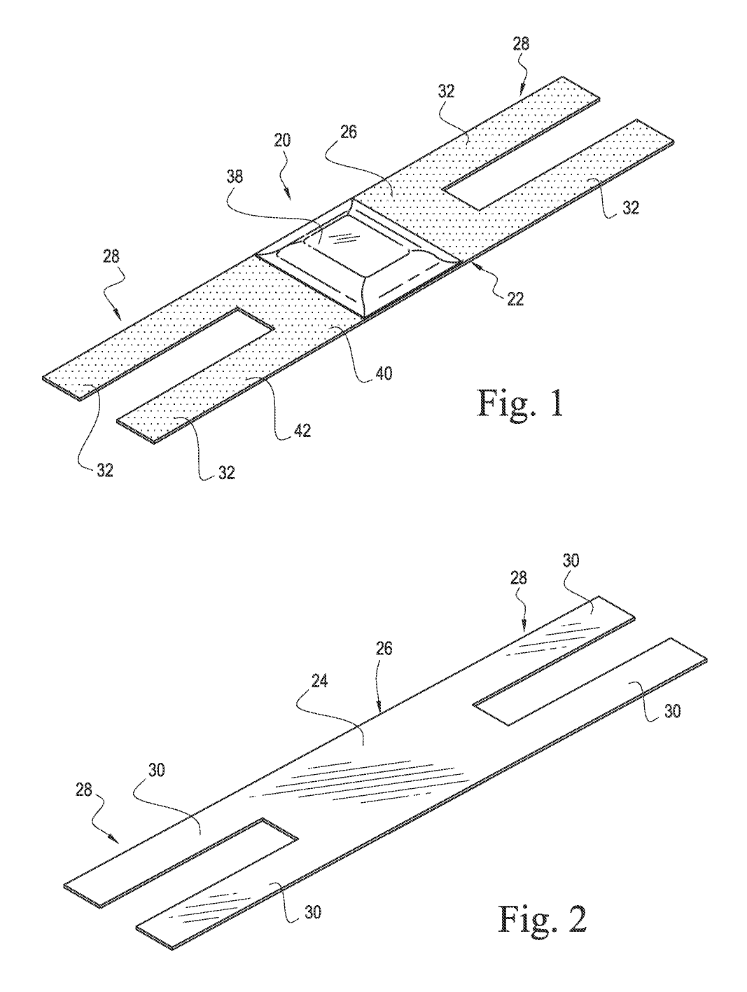

Referring now to FIGS. 1 and 2, details of the intravenous splint cover 20 and methods according to an embodiment of the present invention are now discussed in greater detail. The intravenous splint cover 20 may include a main body 22. The main body 22 is illustratively defined by a medial portion of the intravenous splint cover 20. As illustrated in FIGS. 1 and 2, the main body 22 of the intravenous splint cover 20 may include a bottom portion 26 and a top portion 24.

As further illustrated in FIGS. 1 and 2, the intravenous splint cover 20 may include a pair of opposing securing members 28 that are connected to and extend outwardly from the main body 22. Each of the pair of the opposing securing members 28 may also have a bottom portion 32 and a top portion 30. The intravenous splint cover 20 according to an embodiment of the present invention may also include a splinting member 38 that is carried by the bottom portion 26 of the main body 22. The splinting member 38 illustratively extends outwardly from the main body 22 of the intravenous splint cover 20 to create an elevated portion of the main body. More specifically, and with reference to FIG. 1, the splinting member 38 is also positioned along a medial portion of the main body 22 and, in the illustrated embodiment, along a medial portion of the IV splint cover 20. Those skilled in the art will appreciate, however, that the positioning of the splinting member 38 should not be limited to a medial portion of the IV splint cover 20 as it is contemplated that a length of the securing members 28 may differ. Additional details regarding the shape and positioning of the splinting member 38 and the securing members 28 of the IV splint cover 20 will be provided below.

The main body 22 and the securing members 28 of the IV splint cover 20 according to an embodiment of the present invention may advantageously be integrally formed as a monolithic unit. This advantageously enhances ease of manufacture, and decreases costs associated with manufacture. Those skilled in the art will appreciate, however, that the IV splint cover 20 according to the present invention may advantageously be manufactured by connecting the securing members 28 to the main body 22 so that the securing members and the main body are separate structural members that are joined together. The description of the IV splint cover 20 being integrally formed as a monolithic unit should not be read as limiting, but is meant to illustrate one advantageous configuration of the present invention.

In one embodiment of the IV splint cover 20 according to the present invention, the bottom portions 32 of the pair of opposing securing members 28 may have an adhesive material 42 applied thereto. Similarly, the bottom portion 26 of the main body 22 that does not carry the splinting member 38 may have an adhesive material 40 applied thereto. It is important for the reader to appreciate that the embodiment of the IV splint cover 20 of the present invention that uses an adhesive 40, 42 in connection with the securing members 28 and the main body 22 does not have any adhesive applied to the portion of the main body where the splinting member 38 is positioned. This advantageously allows for the IV site to be readily visualized by a medical professional, for example, or anyone that is may have a need to visualize the IV site on the patient. This is also advantageous to enhance comfort of the patient while the IV splint cover 20 according to an embodiment of the present invention is applied to the patient. More specifically, removal of the IV splint cover 20 from the extremity of the patient upon completion of the IV treatment may be made more comfortable when an adhesive is not included on the splinting member 38. Additional details regarding the advantages of being able to readily visualize the IV site of the patient when using the IV splint cover 20 according to embodiments of the present invention are discussed below.

The adhesive material that may be applied to the bottom portions 32 of the securing members 28 may have stronger adhesive properties than the adhesive material that is applied to the bottom portion 26 of the main body 22. This may advantageously enhance the ability of the IV splint cover 20 to be readily secured to the IV site on the patient. More specifically, providing a stronger adhesive on the securing members 28 may advantageously allow for the securing members to be readily secured to the skin of the patient when using the IV splint cover 20 according to an embodiment of the present invention. Further, using an adhesive on the portion of the main body 22 (the portion that does not include the splinting member 38) that is weaker than the adhesive used on the securing members 28 may advantageously enhance comfort of the patient when the IV splint cover 20 is applied to the IV site. In other words, when removing the IV splint cover 20, a patient may experience less discomfort adjacent to the IV site through the use of a weaker adhesive on the portions of the main body 22 where the splinting member 38 is not positioned.

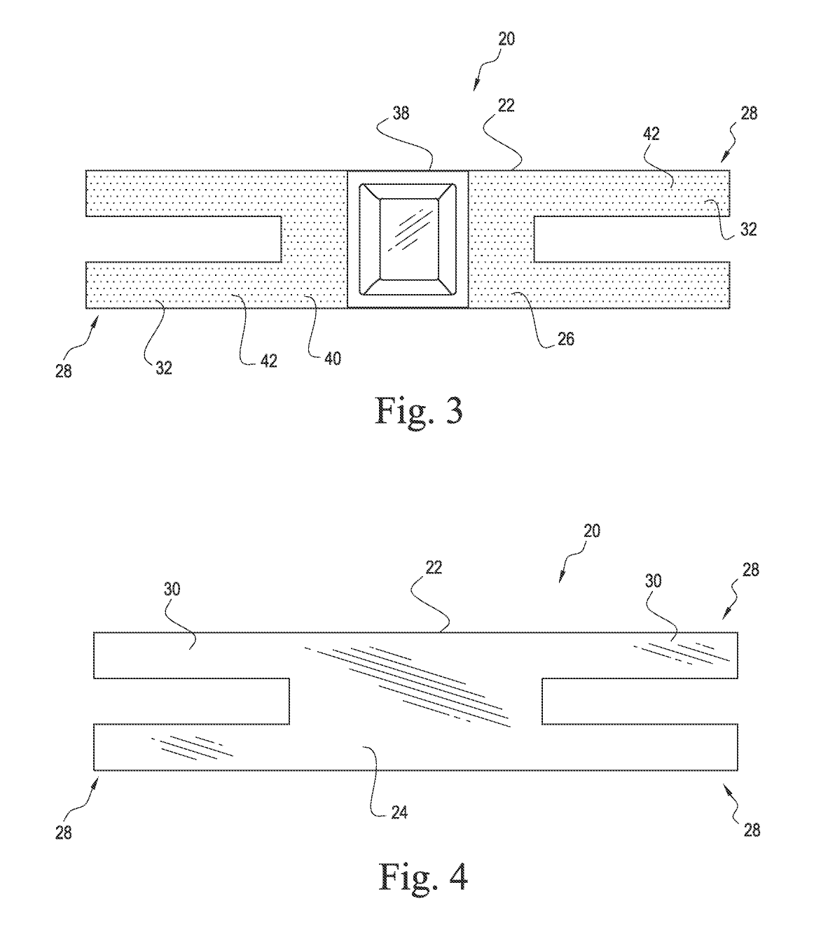

The splinting member 38 may extend along the bottom portion of the main body substantially between the entire width thereof. More specifically, and as perhaps best illustrated in FIGS. 1 and 3, substantially the entire splinting member 38 may extend the entire width of the main body, but the elevated portion of the splinting member may taper from an elevations that is about equal to or higher than the bottom portion of the main body, to a pinnacle thereof. Accordingly, and as perhaps best illustrated in FIG. 1, the splinting member may advantageously taper downwardly from the pinnacle to the main body on four sides in a trapezoidal shape. Those skilled in the art will readily appreciate, however, that any description of the shape of the splinting member 38 is not meant to be limiting in any way and, instead, are provided as examples for clarity. The splinting member 38 may have a rectangular shape, as shown in the figures. A rectangular shaped splinting member may 38 advantageously be easy to manufacture and package, and may provide enhanced convenience in storing and transporting, i.e., in boxes and with respect to shelf space. A rectangular shaped splinting member 38 may also be advantageous to overlap with an IV site plastic cover. Such covers are generally clear, and have a rectangular shape. Other alternate shapes of the splinting member 38 may, for example, be circular, square, ovular, or any other type of shape suitable for being carried by a medial portion of the main body 22.

The splinting member 38 may be made of a substantially soft and pliable material. For example, the splinting member 38 may include a foam type of material, or a cotton splint material. Cotton materials may be preferable to form the splinting member as it may provide enhanced rigidity, and may be more suitable for patients that have allergies to various materials. The use of such material advantageously enhances comfort of the IV splint cover 20 when applied to the IV site of the patient. Such material may also advantageously have certain absorption properties that can be advantageous to soak minor amounts of blood, fluid, or medication leaks that may be associated with starting the IV. Comfort of the patient is one of the several advantages provided by the IV splint cover 20 according to the present invention, and those skilled in the art will appreciate that the splinting member 38 may be made of any other similar material that provides enhanced comfort to the patient when applied the IV site.

As perhaps best illustrated in FIG. 1, each of the pair of opposing securing members 28, i.e., each of the securing members on either side of the main body 22 of the IV splint cover 20, may include a pair of securing members. More specifically, and with specific reference to FIGS. 1 and 2, the securing members 28 may be spaced apart from one another and extend outwardly from the main body 22 substantially parallel to one another. The securing members 28 on either end of the main body 20 may be considered symmetrical to one another. The present invention contemplates, however, that the securing members 28 on either end of the main body 22 of the IV splint cover 20 may have different lengths or may have similar lengths. Application of the of the IV splint cover 20 may, in some instances be facilitated if the securing members 28 on one end of the main body 22 have a length that is greater than the securing members on the opposing end of the main body. Again, this may be an optional feature, and the skilled artisan will appreciate that any length of the securing members 28 of the IV splint cover 20 according to an embodiment of the present invention is contemplated by the present invention.

Although the embodiment of the IV split cover 20 illustrated in the appended figures show the use of a pair of securing members 28 on either side of the main body 22, those skilled in the art will appreciate that any number of securing members, configured in any way, may be provided while still carrying out the many different goals, features and advantages according to the present invention. For example, the illustrated embodiment of the IV splint cover 20 shows a pair of securing members 28 extending parallel to one another outwardly from each side of the main body 20, but those skilled in the art will appreciate that the securing members 28 do not need to necessarily extend parallel to one another out from the main body. In fact, the securing members 28 may extend in any direction, e.g., diagonally. Further, although a pair of securing members 28 are illustrated on either side of the main body 22, the present invention contemplates that more than two securing members may be used to carry out the many different goals and features of the present invention. More specifically, it is contemplated that three securing members 28 may extend outwardly from the main body 22 of the IV splint cover 20. Such a configuration may advantageously provide enhanced security of the IV splint cover 20 according to the present invention when applied to an IV site of a patient. It should be noted, however, that the illustrated embodiment of the IV splint cover 20 does advantageously provide for enhanced security of the IV site of the patient while simultaneously providing the patient with ease of mobility of the extremity where the IV site is positioned.

The material that the IV splint cover 20 according to an embodiment of the present invention may be made of, i.e., the main body 22 and the securing members 28, may be a disposable material that is preferably biodegradable. Such materials are readily known in the art. Using such material is advantageous to reduce waste. Those skilled in the art will appreciate that the main body 22 and the securing members 28 of the IV splint cover 20 may, however, be made of any type of material, while still readily achieving the many different goals and features according to the present invention. It is also preferable that the material of the IV splint cover 20 be substantially flexible. Using such material enhances application of the IV splint cover 20 to the IV site of the patient. As will be discussed in greater detail below, application of the IV splint cover 20 to the IV site of the patient can be accomplished in a one step process. This one step process is enhanced when using a flexible material to form the main body 22 and the securing members 28 of the IV splint cover 20.

It is also preferable that all material used to construct the IV splint cover 20 according to the present invention is made of a hypoallergenic material. Although it is readily understood that constructing the IV splint cover 20 out of such material may be a more costly approach, it is preferable to provide an IV splint cover that can be readily used for all patients. Those skilled in the art, however, will appreciate that the IV splint cover 20 may be readily made of any material, and that use of a hypoallergenic material is an available option contemplated by the present invention. The IV splint cover 20 according to the present invention may also be made of a latex free material to further account for possible allergies of a patient. Again, those skilled in the art will appreciate that any material may be used to construct the IV splint cover 20, but the present invention readily contemplates use of such materials to account for possible allergies that patients may have. Although many patients may be allergic to certain types of materials, the present invention contemplates using paper types of materials to construct the IV splint cover 20.

*The material that may be used to construct the IV splint cover 20 may also have a color that is similar to the skin tone of the patient. This is advantageous for many reasons. Although it may seem that having an IV splint cover 20 with a color that is similar to the skin tone of the patient is merely aesthetic, those skilled in the art will appreciate that using an IV splint cover 20 having a color similar to the skin tone of the patient is advantageous as it can hide the IV splint cover from view of the patient. This may be especially advantageous when using the IV splint cover 20 on patients that may be easily confused. Such patients may desire to rip the IV out, and use of the IV splint cover 20 according to the present invention may hide the IV from site of such patients, thereby detracting attention from it.

It may also be advantageous to provide a material for the main body 22 and the securing members 28 that is waterproof or water resistant. This advantageously allows a patient to bath or shower while the IV splint cover 20 is applied to the IV site. Accordingly, the IV splint cover 20 according to embodiments of the present invention not only allows the patient's extremity to remain mobile while in use, but also advantageously enhances the ability of the patient to engage in other routine daily activities while IV medical treatments are being administered. Further, the IV splint cover 20 is advantageous for patients that are receiving multiple IV treatments, i.e., use of the IV splint cover prevents immobilization of the patient's extremities, which can be especially advantageous for patients that may have IV's in two different extremities.

As perhaps best illustrated in FIG. 10, the IV splint cover 20 according to the present invention may further include adhesive cover members 44 that may be removeably positioned over the main body 22 and the securing members 28. The adhesive cover members 44 are positioned to overlie the bottom portion 26 of the main body 22 and the bottom portion 32 of the securing members 28. Accordingly, the adhesive cover members 44 may readily protect the adhesive from exposure when not in use to ensure that the adhesive is remains strong enough to be applied to a patient. As further illustrated in FIG. 10, the adhesive cover members 44 may be provided by a pair of adhesive cover members. A first one of the pair of adhesive cover members 44 may be positioned to extend from an end portion of one of the securing members 28 to a medial portion of the main body 22 of the IV splint cover 20, and a second one of the pair of adhesive cover members 44 may extend form an end portion of the opposing securing member to the medial portion of the main body. Accordingly, the present invention contemplates that end portions of the adhesive cover members 44 may somewhat overlap adjacent the medial portion of the main body 22 so that the adhesive cover members can be readily removed when used.

The adhesive cover members 44 may be made of a paper material, a paper material having a plastic coating, or a plastic material to cover the portions of the main body 22 and the securing members 28 that have adhesive material applied thereto. The use of such materials for the adhesive cover members 44 advantageously allows for the adhesive cover members to be readily connected to the adhesive portions of the main body 22 and the securing members 28 in a manner that allows the adhesive to remain protected from the elements, and that allows for the cover members 44 to be readily removed from the adhesive portions of the IV splint cover 20.

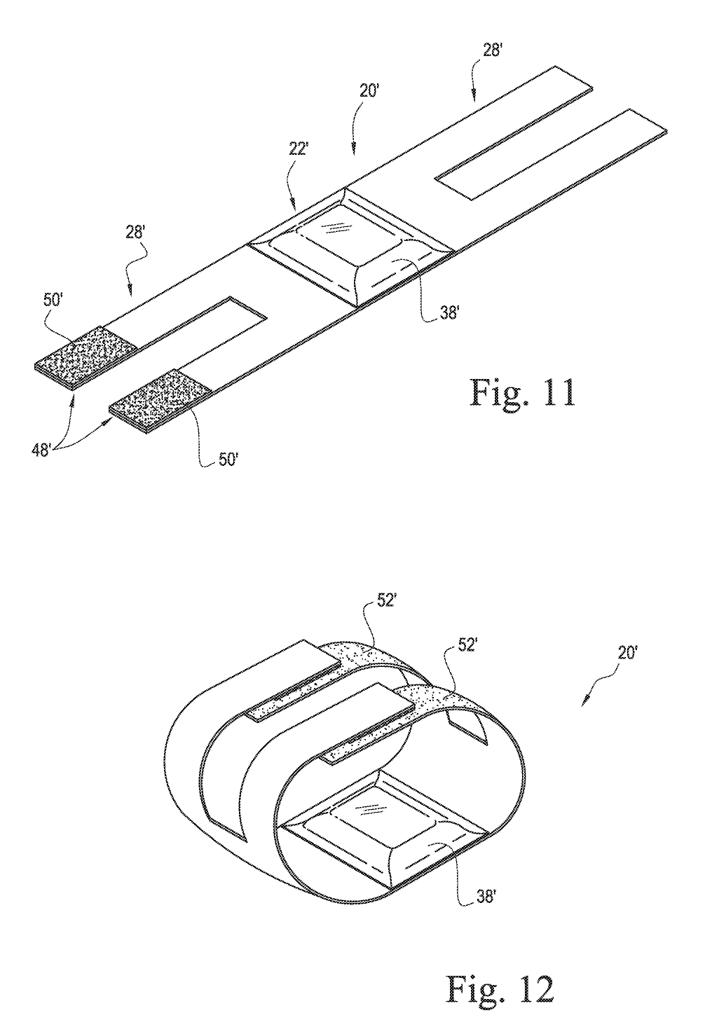

Referring now additionally to FIGS. 11 and 12, an alternate embodiment of the IV splint cover 20′ is now discussed in greater detail. More specifically, the IV splint cover 20′ may include a pair of opposing securing members 28′ that are connected to and extend outwardly from the main body 22′. In this embodiment of the IV splint cover 20, fasteners 48′ may be carried by end portions of each of the pair of securing members 28′. The fasteners 48′ may include fasteners 50′ positioned on the bottom side of the securing members 28′ and fasteners 52′ positioned on the top side of the securing members. The fasteners 48′ may be provided by hook and loop fasteners, as understood by those skilled in the art. The other elements of this embodiment of the IV splint cover 20′ not specifically described herein are similar to those of the first embodiment of the IV splint cover 20 described above, are labeled with prime notations, and require no further discussion herein.

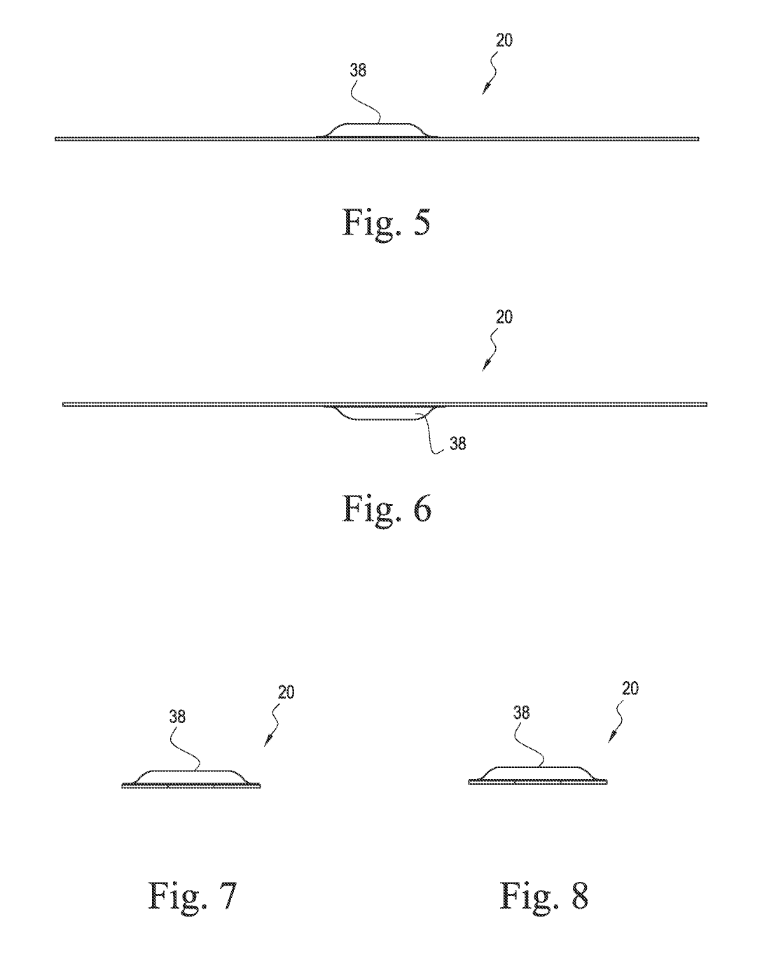

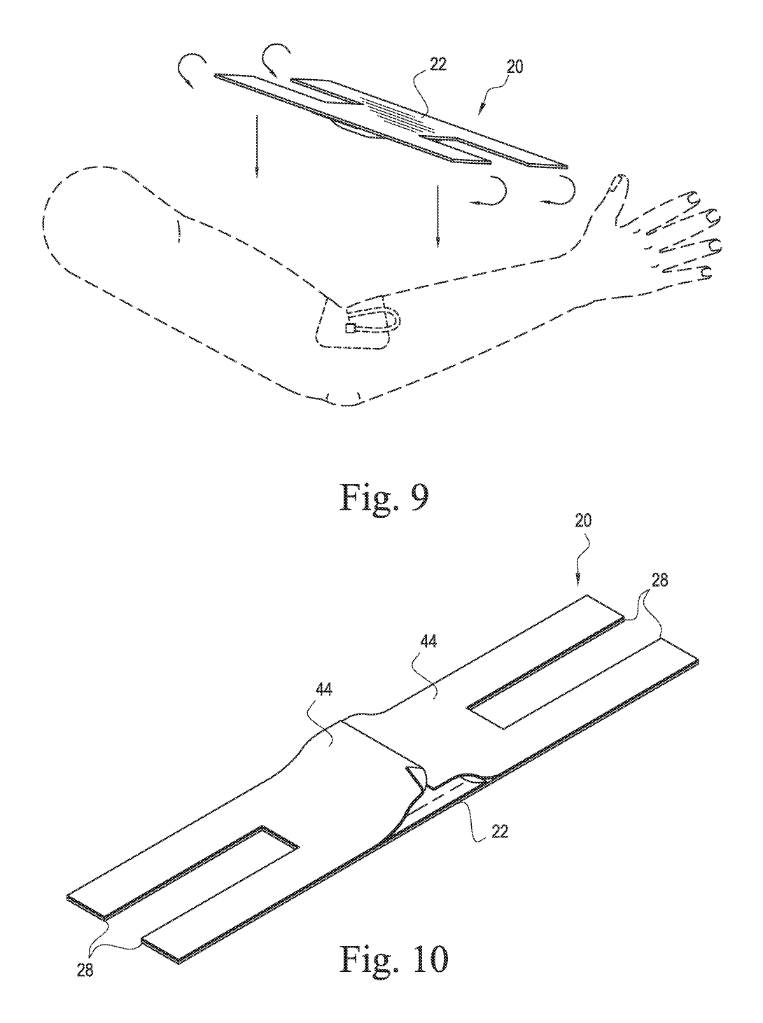

Referring now back to FIGS. 1 through 10, use of the IV splint cover 20 according to an embodiment of the present invention is now described in greater detail. When using the IV splint cover 20 according to an embodiment of the present invention, a user, such as, for example, a medical professional, may remove the IV splint cover 20 from a package. The packaging that contain the IV splint cover is preferably a sterilized material to ensure that the IV splint cover 20 remain sterile during transport. This advantageously reduces the risk of infection to the patient when using the IV splint cover 20. Those skilled in the art will appreciate that although the packaging that houses the IV splint cover 20 is described herein, the function of the present invention can be readily carried out without the use of such packaging.

After the IV splint cover 20 according to the present invention has been removed from the packaging, the medical professional may remove the cover members 44 positioned to overlie the bottom portion 26 of the main body 22 and the bottom portion 32 of the securing members 28. Removal of the cover members 44 advantageously exposes the adhesive material 40, 42 positioned on the bottom portion 26 of the main body 22 and the bottom portion 32 of the securing members 28. Removal of the cover members 44 also advantageously exposes the splinting member 38. After the bottom portion of the main body 28 has been exposed, the medical professional may readily apply the IV splint cover 20 to the IV site of the patient.

Those skilled in the art will appreciate that the medical professional may completely remove the cover members 44 or may, alternatively, only remove enough of the cover members to expose the splinting member 38. As described below, the securing members can be applied directly to the skin of the patient adjacent to the IV site, if the cover members are fully removed, or the cover members 44 can be removed as the securing members are applied to the skin of the patient. In other words, the process of removing the cover members 44 and applying the securing members 28 to the skin of the patient can be a substantially simultaneous process. This advantageously allows for the adhesive material on the bottom portion 26 of the main body and on the bottom portion 32 of the securing members 28 to remain covered until almost the instant that the main body and the securing members are to be applied to the IV site.

When applying the IV splint cover 20 to the IV site of the patient, the medical professional, after starting the IV, may apply the splinting member 38 over the IV site. Application of the splinting member 38 to the IV site may advantageously prevent the IV catheter from bending when the patient bends their extremity. More particularly, the splinting member 38, after being applied over the IV site, keeps the IV catheter from bending, but advantageously allows the extremity of the patient to bend, so that the patient can be comfortable while receiving IV treatments and can enjoy other routine daily activities. Thereafter, the securing members 28 may be stretched over the extremity of the patient, and the adhesive material 40, 42 on the main body 22 and the securing members 28 may be applied to the skin of the user. The splinting member 38 advantageously secures the IV site so that the IV does not come out while the patient is receiving IV treatments. Further, application of the IV splint cover 20 to the extremity of the patient where the IV is located advantageously secures the IV while not immobilizing the patient's extremity. In other words, the patient may still readily move and bend the extremity where the IV is located while the IV remains secured.

The configuration of the IV splint cover 20 according to embodiments of the present invention advantageously allows for application of the IV splint cover in a one step process. This step can be described as simply applying the splinting member to the IV site while connecting the securing members 28 to the skin of the patient adjacent to the IV site.

The configuration of the IV splint cover 20 according to the present invention also advantageously provides the medical professional with the ability to readily connect the IV splint cover 20 to the IV site of the patient using one hand, which may sometimes be necessary in many medical settings. For example, after applying the splinting member 38 to the IV site of the patient, the medical professional may hold the splinting member securely on the IV site using a thumb, for example, and may move the securing members 38 over the side portions of the extremity (to either side of the IV site) so that the adhesive portions on the main body 22 of the splint cover and on the securing members contact the skin of the patient. After the IV splint cover 20 according to this embodiment of the invention is positioned over the IV site of the patient, and since adhesive is not applied to portions of the main body 22 where the splinting member 38 is positioned, the IV site may be readily visualized by the medical professional when the IV splint cover is applied.

It is preferable when applying the IV splint cover 22 to the IV site on the patient, that the securing members 28 overlap one another at end portions thereof. More specifically, it is preferable for the medical professional to stretch the securing members 28 a distance suitable for the securing members to overlap one another during application. It is also preferable for the securing members 28 to be applied to the skin of the user adjacent the joint on the patient where the IV site is positioned. This enhances mobility of the patient when the IV splint cover 20 is positioned over the IV site.

Referring now back to the embodiments of the IV splint cover 20′ illustrated in FIGS. 11 and 12, another embodiment of the method of using the IV splint cover 20′ is now described in greater detail. In this embodiment, the fasteners 48′ positioned on the bottom portion of the securing members 50′ and on the top portion of the securing members 52′ may be configured to connect to one another. As described above, the fasteners 48′ may be provided by hook and loop fasteners. Accordingly, and with reference to the illustrated embodiment of the IV splint cover 20′, the securing members 28′ that extend outwardly from a first side of the main body 22′ may have fasteners 50′ positioned on a bottom portion thereof, and the securing members extending from the opposing side of the main body may have fasteners 52′ positioned on a top portion thereof. After the medical professional has applied the splinting member 38′ to the IV site, the securing members may be stretched over the extremity of the patient, and the securing members 28′ may be connected to one another so that the fasteners 48′ may be connected to one another.

Some additional features of the IV splint cover 20 are now described in greater detail. One option that is contemplated by the present invention is to make the IV splint cover 20 child friendly. This can be accomplished by positioning indicia that is child related on the top portion of the main body 22 and the securing members 28. The indicia may, for example, be directed to children's characters, or any other type of indicia that may be pleasing to children. This advantageously directs children's attention away from the IV site, and may focus the children's attention on the characters that are printed on the IV splint cover 20. The IV splint cover 20 according to the present invention also contemplates that the main body 22 and securing members 28 may be very brightly colored, which may also detract a child's attention from the IV site.

Additional advantages are apparent with respect to the IV splint cover 20 according to the present invention. For example, use of the IV splint cover results in a substantial decrease in costs associated with medical care. This is realized from costs saved in additional medical supplies associated with reinsertion of the IV, and also from a decrease in labor costs associated with the medical staff that would otherwise need to reinsert the IV. There is also an inherent cost associated with patient comfort. Patients that do not require reinsertions of IV's are generally more comfortable, more satisfied, and can generally heal quicker, thereby decreasing inherent medical costs associated with prolonged recovery times.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. An intravenous splint cover comprising:
a main body having a bottom portion and a top portion;
a pair of opposing securing members connected to and extending outwardly from the main body and having a bottom portion and a top portion;
a splinting member carried by the bottom portion of the main body and extending outwardly from the main body to create an elevated portion of the main body;
wherein the bottom portions of the pair of opposing securing members have an adhesive material applied thereto;
wherein the bottom portion of the main body that does not carry the splinting member has an adhesive material applied thereto;
wherein the splinting member extends along the bottom portion of the main body substantially between an entire width thereof, and is carried by a medial portion of the main body; and
wherein the splinting member does not carry an adhesive material applied thereto.

2. An intravenous splint cover according to claim 1 wherein each of the pair of opposing securing members comprises a respective pair of spaced apart securing members; and wherein each of the respective pair of spaced apart securing members extend substantially parallel to one another.

3. An intravenous splint cover according to claim 1 wherein the splinting member is raised along a medial portion of the main body and tapers downwardly towards side portions of the main body.

4. An intravenous splint cover according to claim 1 further comprising an adhesive cover member to be removeably positioned over the main body, the securing members and the splinting member.

5. An intravenous splint cover according to claim 4 wherein the adhesive cover member comprises a pair of adhesive cover members; and wherein a first one of the pair of adhesive cover members extends from an end portion of one of the securing members to a medial portion of the splinting member; and wherein a second one of the pair of adhesive cover members extends from an end portion of a second one of the securing member to the medial portion of the splinting member.

6. An intravenous splint cover according to claim 1 wherein the adhesive material that is applied to the bottom portions of the securing members has stronger adhesive properties than the adhesive material that is applied to the main body.

7. An intravenous splint cover according to claim 1 wherein the main body and the pair of opposing securing members are integrally formed as a monolithic unit.

8. An intravenous splint cover according to claim 1 wherein the main body and the pair of opposing securing members comprise at least one of a substantially flexible material, a biodegradable material, a hypoallergenic material, and a latex-free material.

9. An intravenous splint cover comprising:
a main body having a bottom portion and a top portion;
a pair of opposing securing members connected to and extending outwardly from the main body and having a bottom portion and a top portion;
a splinting member carried by the bottom portion of the main body and extending outwardly from the main body to create an elevated portion of the main body, the splinting member being raised along a medial portion of the main body and tapering downwardly towards side portions of the main body;
fasteners carried by end portions of each of the pair of securing members;
wherein the splinting member extends along the bottom portion of the main body between an entire width thereof, and is carried by a medial portion of the main body; and
wherein the splinting member does not carry an adhesive material applied thereto;
wherein each of the pair of opposing securing members comprises a respective pair of spaced apart securing members; and
wherein each of the respective pair of spaced apart securing members extends parallel to one another.

10. An intravenous splint cover according to claim 9 wherein the fasteners are hook and loop fasteners.

11. An intravenous splint cover according to claim 10 wherein a portion of the bottom portion of the securing members includes hook and loop fastener; and wherein a portion of the top portion of the securing members and the main body includes hook and loop fasteners.

12. An intravenous splint cover according to claim 11 wherein the securing members are fastened to at least one of the opposing securing member and the main body after the splinting member is positioned over an intravenous site.

13. An intravenous splint cover according to claim 9 wherein the main body and the pair of opposing securing members are integrally formed as a monolithic unit.

14. An intravenous splint cover according to claim 9 wherein the main body and the pair of opposing securing members comprise at least one of a substantially flexible material, a biodegradable material, a hypoallergenic material, and a latex-free material.

15. A method of using an intravenous splint cover that includes a main body having a bottom portion and a top portion, a pair of opposing securing members connected to and extending outwardly from the main body and having a bottom portion and a top portion, and a splinting member carried by the bottom portion of the main body and extending outwardly from the main body to create an elevated portion of the main body, wherein the splinting member does not carry an adhesive material applied thereto and the main body and the pair of opposing securing members being integrally formed as a monolithic unit the method comprising:
exposing the bottom portions of the main body, the pair of opposing securing members and the splinting member;
applying the splinting member to an intravenous site on a patient; and
securing the splinting member to the intravenous site by securing the securing members to at least one of the patient, the main body and the opposing securing member so that an extremity of the patient where the IV is positioned is not immobilized and so that the intravenous site is accessible.

16. A method according to claim 15 wherein the bottom portions of the pair of opposing securing members have an adhesive material applied thereto;
wherein the bottom portion of the main body that does not carry the splinting member has an adhesive material applied thereto;
wherein the splinting member extends along the bottom portion of the main body substantially between an entire width thereof, and is carried by a medial portion of the main body; and
wherein securing the splinting member to the intravenous site further comprises adhesively securing the bottom portions of the securing members to at least one of the patient, the main body and the opposing securing member.

17. A method according to claim 16 wherein the intravenous splint cover further comprises an adhesive cover member to be removeably positioned over the main body, the securing members and the splinting member; and
wherein exposing the bottom portions of the main body, the pair of opposing securing members and the splinting member comprises removing the adhesive cover member.

18. A method according to claim 15 wherein the intravenous splint cover further comprises fasteners carried by end portions of each of the pair of securing members.

19. A method according to claim 18 wherein the fasteners are hook and loop fasteners;
wherein a portion of the bottom portion of the securing members includes hook and loop fastener;
and wherein a portion of the top portion of the securing members and the main body includes hook and loop fasteners;
and wherein securing the splinting member comprises fastening at least one of the opposing securing member to at least one of the main body and the opposing securing member after the splinting member is positioned over an intravenous site.

20. A method according to claim 15 wherein the main body and the pair of opposing securing members comprise at least one of a substantially flexible material, a biodegradable material, a hypoallergenic material, and a latex-free material.
